(19) United States
(12) Patent Application Publication
(10) Pub. No.: US 2011/0002893 A1
(43) Pub. Date: Jan. 6, 2011

(54) DIAGNOSIS AND TREATMENT OF CONGENITAL HEART DEFECTS USING NELL1

(75) Inventor: Cymbeline T. Culiat, Oak Ridge, TN (US)

Correspondence Address: UT-Battelle, LLC;Office of Intellectual Property, One Bethal Valley Road, 4500N, MS-6258, Oak Ridge, TN, 37831, US

(73) Assignee: UT-BATTELLE, LLC, Oak Ridge, TN (US)

(21) Appl. No.: 12/611,292

(22) Filed: Nov. 3, 2009

Related U.S. Application Data
(60) Provisional application No. 61/110,651, filed on Nov. 3, 2008.

(51) Int. Cl. A61K 35/12; C12Q 1/68; C12Q 1/02; A61K 38/02; A61K 31/7088; G01N 33/50; A61P 9/00

(52) U.S. Cl. 424/93.7; 435/6; 435/29; 514/16.4; 514/44 R; 436/86

(57) ABSTRACT

The present invention provides diagnostic methods for detecting congenital heart defects, or increased risk thereof, based on the Nell1 gene, RNA and protein. The methods include obtaining a biological sample and assessing the presence of a mutation in the Nell1 gene, RNA or protein. The presence of a mutation in the Nell1 gene, RNA or protein can be assessed by determining the levels of Nell1 gene, RNA or protein in the biological sample. The present invention further provides therapeutic methods for treating congenital heart defects based on the Nell1 gene. RNA and protein.

This application asserts the priority of U.S. provisional application Ser. No. 61/110,651 filed Nov. 3, 2008, the specifications of which are hereby incorporated by reference in their entirety

### STATEMENT REGARDING FEDERALLY SPONSORED RESEARCH

This invention was made with Government support under Contract No. DE-AC05-00OR22725 between the United States Department of Energy and UT-Battelle, LLC. The Government has certain rights in this invention.

### FIELD OF THE INVENTION

The present invention relates in general to diagnostic methods for detecting, and therapeutic methods for treating, congenital heart defects based on the Nell1 gene, RNA and protein. These methods capitalize on the cell signaling pathway mediated by Nell1 in the proper formation of heart structures, thus imparting normal heart functions.

### BACKGROUND OF THE INVENTION

Congenital heart defects (CHD) are heart defects present at birth and are often structural abnormalities that cause arrhythmia or heart muscle malfunction. The wide spectrum of CHD represents a major cause of infant mortality and serious health problems in young children. In addition to its critical impact on normal fetal and infant development. CHD can go undetected in early childhood and become manifested later as life-threatening or debilitating heart conditions in adult patients, such as valve problems, transposition disorders, septal defects and blood vessel and artery problems. Thus, there is a need for early diagnosis, proper care and treatment of patients with CHD.

### SUMMARY OF THE INVENTION

In one embodiment, the invention provides a method for detecting a congenital heart defect in a mammal. The method comprises providing a biological sample from the mammal, wherein said biological sample comprises a Nell1 nucleic acid molecule, and assessing said Nell1 nucleic acid molecule for the presence of a mutation; whereby the presence of a mutation in the Nell1 nucleic acid molecule indicates presence of a congenital heart defect in the mammal.

In another embodiment, the invention provides a method for detecting increased risk for a congenital heart defect in a mammal. The method comprises providing a biological sample from the mammal, wherein said biological sample comprises a Nell1 nucleic acid molecule, and assessing said Nell1 nucleic acid molecule for the presence of a mutation; whereby the presence of a mutation in the Nell1 nucleic acid molecule indicates increased risk for a congenital heart defect in the mammal

In a further embodiment, the invention provides a method for detecting a congenital heart defect in a mammal. The method comprises providing a biological sample for the mammal, wherein said biological sample comprises Nell1 protein, and assessing said Nell1 protein for the presence of a mutation; whereby the presence of a mutation in the Nell1 protein indicates presence of a congenital heart defect in the mammal.

In yet another embodiment, the invention provides a method for detecting increased risk for a congenital heart defect in a mammal. The method comprises providing a biological sample for the mammal, wherein said biological sample comprises Nell1 protein, and assessing said Nell1 protein for the presence of a mutation; whereby the presence of a mutation in the Nell1 protein indicates increased risk for a congenital heart defect in the mammal

In yet a further embodiment, the invention provides a method for treating a congenital heart defect in a mammal in need thereof. The method comprises administering an effective amount of Nell1 protein to the mammal.

In another embodiment, the invention provides a method for treating a congenital heart defect in a mammal in need thereof. The method comprises administering to the mammal a nucleic acid coding for a Nell1 protein.

For a better understanding of the present invention, together with other and further advantages, reference is made to the following detailed description, and its scope will be pointed out in the subsequent claims.

### BRIEF DESCRIPTION OF THE FIGURES

FIG. 1. Expression of the Nell1 gene in developing heart.

FIG. 2. Spectrum of heart valve defects resulting from the absence of Nell1 function.

FIG. 3. Abnormalities in number and shape of heart valve leaflets resulting from the absence of Nell1 function

### DETAILED DESCRIPTION OF THE INVENTION

The invention is based on the novel discovery by the inventor that the presence of a mutation in the nucleic acid sequence of Nell1 and/or the amino acid sequence of Nell1 protein is associated with congenital heart defects.

Throughout this specification, parameters are defined by maximum and minimum amounts. Each minimum amount can be combined with each maximum amount to define a range.

### Congenital Heart Defect

The term “congenital heart defect” as used herein refers to an abnormality of the heart or a great vessel that is present from birth. The term “great vessel” as used herein refers to a primary blood vessel. Examples of primary blood vessels include, but are not limited to superior vena cavae, inferior vena cavae, pulmonary artery (e.g., left pulmonary artery, right pulmonary artery, pulmonary trunk, etc.), aorta, pulmonary veins (e.g., right superior pulmonary vein, left superior pulmonary vein, right inferior pulmonary vein, left inferior pulmonary vein, etc.). These also include the vessels in the heart's own circulatory system, such as the major coronary arteries.

The health effects of a congenital heart defect can manifest at any time in the lifespan of a mammal. For example, the congenital heart defect can manifest at birth; soon after birth, such as within one or more weeks after birth, within one or more months after birth, or within one or more years after birth; during childhood, or during adulthood. Manifestations soon after birth are typically associated with the change from fetal to postnatal circulatory patterns (e.g., reliance on the lungs, rather than the placenta, for oxygenation).

Congenital heart defects can be caused by unknown or known factors. The known causes can be of a multifactorial origin, a result of genetic predisposition, and/or environmental factors. Known genetic causes of congenital heart defects includes chromosomal abnormalities such as trisomies 21, 13 and 18, genetic abnormalities such as genetic point mutations, point deletions and other genetic abnormalities as seen in syndromes such as CATCH 22, familial ASD with heart block, Alagille syndrome. Noonan syndrome, etc.

Environmental factors that can cause congenital heart defects include infections (e.g., bacteria, viral, etc.) during pregnancy; exposure to drugs (e.g., alcohol, hydantoin, lithium and thalidomide, etc.), chemicals, or radiation, during pregnancy; and maternal illness (e.g., diabetes mellitus, phenylketonuria, and systemic lupus erythematosus. etc.).

Examples of congenital heart defects in accordance with the aspects of the invention include, but are not limited to, those listed in Table I. These may also include patent ductus arteriosus, lutembaher disease, ostium secundum, ventricular septal defect and patent ductus arteriosus. Fallot's triad, Eisenmenger's complex, partial atrioventricular canal, ostium primum, partial anomalous pulmonary venous connection, ventricular septal defect. Potts and Waterston-Cooley shunts, atrioventricular canal, Ebstein's anomaly, stenosis of lung artery, tricuspid atresia, truncus arteriosus, tetralogy of Fallot, coarctation of aorta and an open arterial channel, total anomalous pulmonary venous connection, transposition of the great arteries, coarctation of the aorta, and aortic stenosis.

TABLE 1
Examples of Congenital Heart Defects

| HEART AND/OR BLOOD | | |
|---|---|---|
| VESSEL DEFECT | DESCRIPTION/DEFINITION | REFERENCES |
| Mitral Valve Prolapse | Valve between left atrium and | Takano H. et al. 2005. Ann Thorac Surg.; |
| (Mitral Regurgitation) | left ventricle does not close | Schwarz U. et al. 2004. Hum Genet; Leier |
| | properly so blood flows back | C. et al. 1980. American College of |
| | into atrium | Physicians. |
| Aortic Regurgitation | Valve between left ventricle and | Price C M et al. 1996. British Journal |
| | aorta does not close properly so | of Anaesthesia; Schwarz U. et al. |
| | blood flows back into ventricle | 2004. Hum Genet |
| Myocardial Ischemia | Restriction in blood supply | Price C M et al. 1996. British |
| | causing damage to heart tissue | Journal of Anaesthesia. |
| Friable Arteries/ | Decreased amount of collagen | Takano H. et al. 2005. Ann Thorac Surg. |
| Vascular Rupture | Fibers; weaker, damage-prone | Price C M et al. 1996. British Journal |
| | arteries | of Anaesthesia. |
| Ventricular Hypertrophy | Enlarged ventricles; increased | Price C M et al. 1996. British Journal |
| | vessel wall thickness | of Anaesthesia. |
| Supravalvular | Narrowing within pulmonary | Price C M et al. 1996. British Journal |
| Pulmonary Stenosis | valve diameter | of Anaesthesia.; Leier C. et al. 1980. |
| | | American College of Physicians. |
| Pulmonary Hypertension | Increased blood pressure in | Leier C. et al. 1980. American |
| | pulmonary vessels | College of Physicians. |
| Increased Vessel Wall | May cause decrease in blood | Kerwin W. et al. 2007. Int J |
| Thickness | pressure | Cardiovasc Imaging. |
| Myxomatous Change | Benign tumor formation in heart | Takano H. et al. 2005. Ann Thorac Surg. |
| Aortic Dilation/Aortic | Over expansion of arterial | Wenstrup R J. et al. 2002. Genet Med. |
| Root Dilation | diameter or aortic root | Zilocchi M. et al. 2007. AJR Am J |
| | | Roentgenol.; Kerwin W. et al. 2007. Int |
| | | J Cardiovasc Imaging; Leier C. et al. |
| | | 1980. American College of Physicians. |
| Arterial Aneurysm | Blood-filled dilation of blood | Zilocchi M. et al. 2007. AJR Am |
| | vessel caused by pathological | J Roentgenol. |
| | disease or wall feebleness | |
| Arterial Occlusion | Blocked or obstructed arteries | Zilocchi M. et al. 2007. AJR Am |
| | | J Roentgenol. |
| Carotid Cavernous | Abnormal duct connects carotid | Zilocchi M. et al. 2007. AJR Am |
| Fistula | artery to porous cavities | J Roentgenol. |
| Patent Ductus Arteriosus | Connection between pulmonary artery | Maeda T. et al. 2002. Intern Med. |
| (PDA) | and aorta remains open straining the | |
| | heart and increasing blood pressure | |
| Septal Defects | Septum or wall division between | Maeda T. et al. 2002. Intern Med. |
| | the atria and ventricles contains | |
| | large opening or “hole.” | |

### Nell1

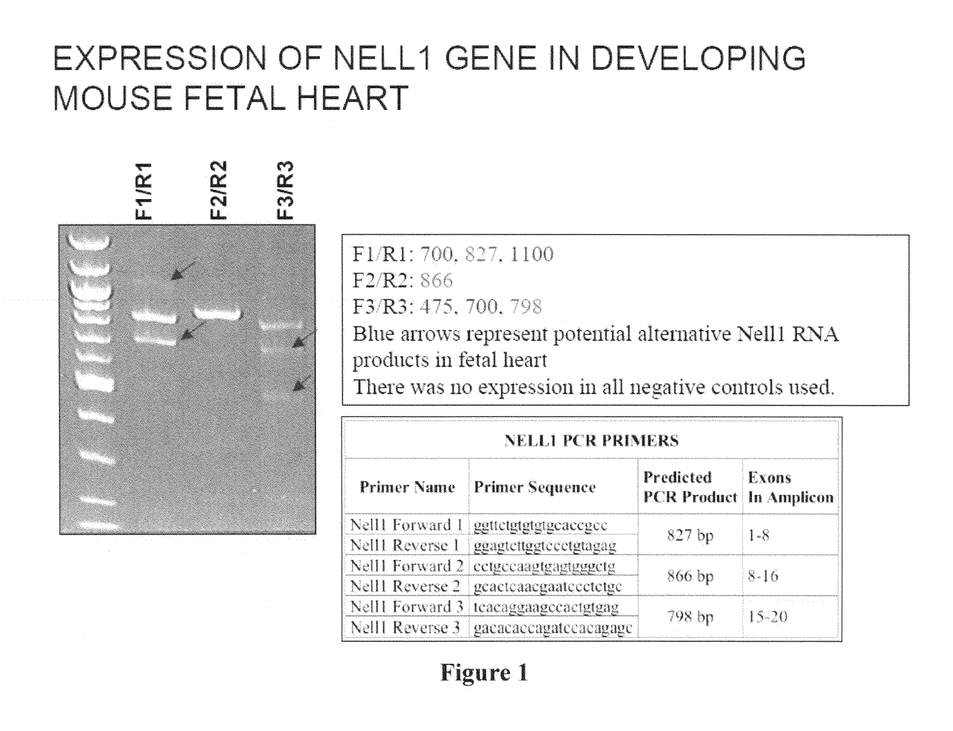

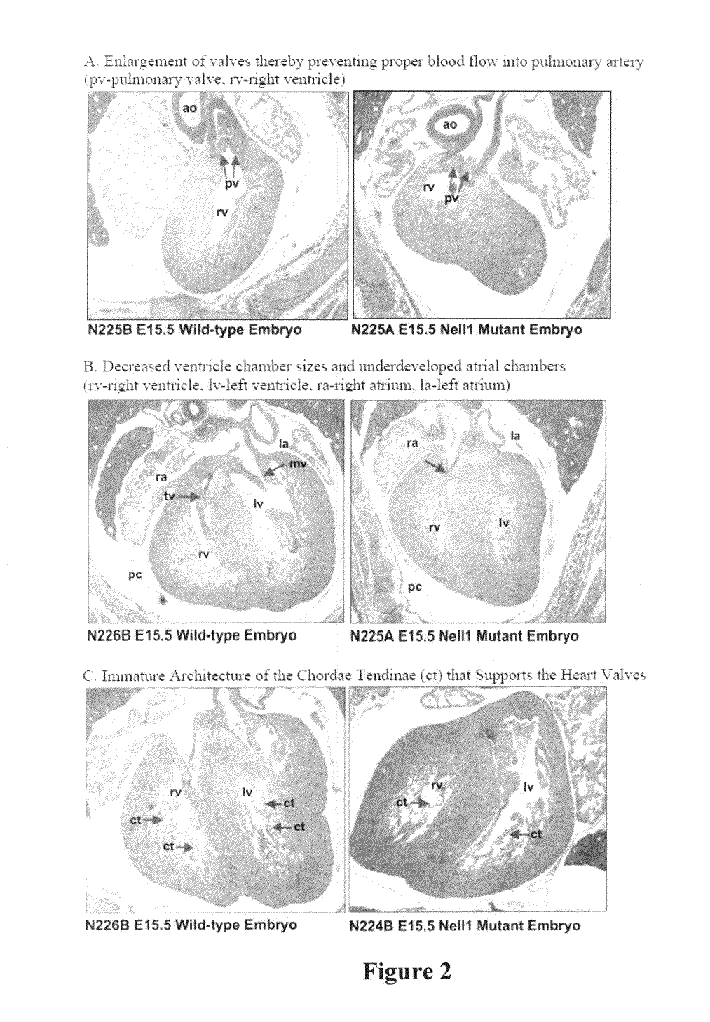

Nell1 protein is a protein kinase C (PKC) β-binding protein. The amino acid sequence of human wild-type Nell1 protein can be found at GenBank Accession No. AAH96102. and is shown in FIG. 1 (SEQ. ID. NO: 1). Due to the degeneracy of the genetic code, an example of a nucleic acid sequence which encodes SEQ. ID. NO: 1 is shown in FIG. 2 (SEQ. ID. NO:2).

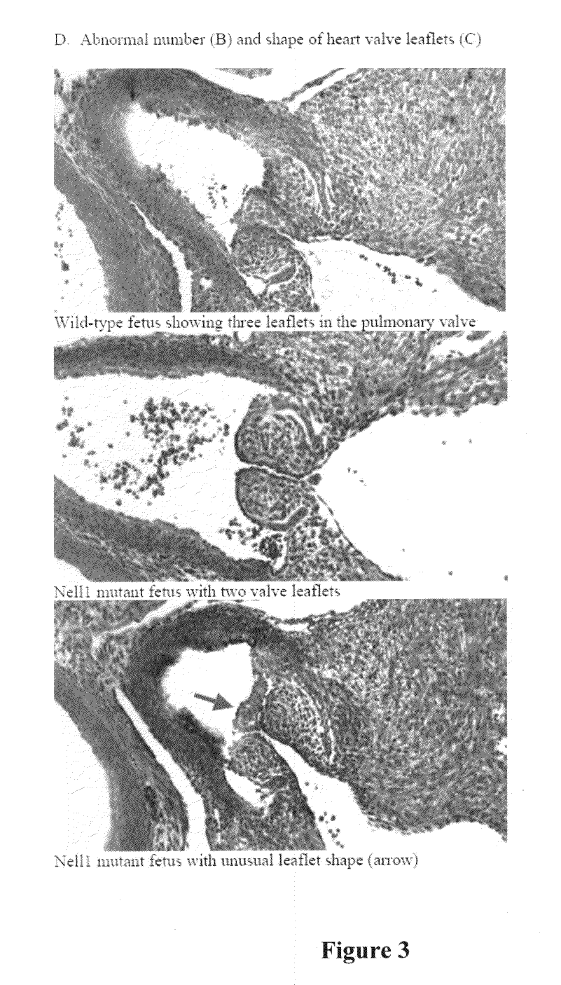

The amino acid sequence of rat wild-type Nell1 protein can be found at GenBank Accession No. NP_{—}112331, and is shown in FIG. 3 (SEQ. ID. NO: 3). An example of a nucleotide sequence which encodes SEQ. ID. NO: 3 is shown in FIG. 4 (SEQ. ID. NO: 4).

The amino acid sequence of mouse wild-type Nell1 protein can be found at GenBank Accession No. NP_{—}001032995. and is shown in FIG. 5 (SEQ. ID. NO: 5). An example of a nucleotide sequence which encodes SEQ. ID. NO: 5 is shown in FIG. 6 (SEQ. ID. NO: 6).

### Method for Detecting, a Congenital Hear Defect or Increased Risk of a Congenital Heart Defect, by Assessing Presence of Mutation in Nell1 Nucleic Acid Molecule

In one aspect, the invention provides a method for detecting a congenital heart defect in a mammal by assessing Nell1 nucleic acid molecules for the presence of a mutation. In another aspect, the invention provides a method for detecting increased risk for a congenital heart defect by assessing Nell1 nucleic acid molecules for the presence of a mutation.

The first step in these methods is to provide a biological sample. The biological sample can be obtained, in the same laboratory in which the method is performed, or in another center and later sent to the laboratory for study. The biological sample contains a Nell1 nucleic acid molecule. The Nell1 nucleic acid molecule can be genomic DNA, RNA, and/or cDNA.

Examples of biological samples containing Nell1 nucleic acid molecules include blood cells, saliva, epithelial cells, fetal cells, etc. The biological sample can he obtained by any method known to those in the art. Suitable methods include, for example, venous puncture of a vein to obtain a blood sample and cheek cell scraping to obtain a buccal sample.

The method can be performed on a fetus. Thus, for prenatal diagnosis, examples of biological samples that contain Nell1 nucleic acid molecules include fetal cells, placental cells, amniotic fluid, or a chorion villus sample. Methods for obtaining a biological sample from a fetus are known to those skilled in the art. For example fetal blood (e.g., cord blood) may be obtained from the umbilical cord by cordocentesis as described in Daffos et al. (Am. J. Obstet Gynecol., 1985, 153:655-660). Alternatively, amniotic fluid can be obtained, for example by amniocentesis (see for example, Marthin et al., Acta. Obstet. Gynecol. Scand., 1997, 76:728-732).

Nucleic acid molecules can be isolated from a biological sample by any method known to those in the art. For example, commercial kits, such as the QIAGEN System (QIAmp DNA Blood Midi Kit, Hilder, Germany) can be used to isolate DNA. The Nell1 nucleic acid molecule is optionally amplified by methods known in the art. One suitable method is the polymerase chain reaction (PCR) method described by Saiki et al., Science 239:487 (1988). U.S. Pat. No. 4,683,195 and Sambrook et al. (Eds.), Molecular Cloning, Third Edition. Cold Spring Harbor Laboratory Press, Cold Spring Harbor, N.Y. (2001). For example, oligonucleotide primers complementary to a nucleotide sequence flanking and/or present in the nucleotide sequence of Nell1 can be used to amplify the nucleic acid molecule.

In one embodiment, the isolated Nell1 nucleic acid molecule is used to assess whether a mutation is present in the Nell1 nucleic acid molecule. The presence of a mutation in a Nell1 nucleic acid molecule can be determined by any method known to those skilled in the art. Such methods include, for example, hybridization of nucleic acid probes, allele-specific polymerase chain reaction (PCR) assays, restriction site digestion and direct sequencing methods. Methods for making and using nucleic acid probes are well documented in the art. For example, see Keller G H and Manak M M. DNA Probes, 2.sup.nd ed., Macmillan Publishers Ltd., England (1991) and Hames B D and Higgins S J. eds., Gene Probes I and Gene Probes 11, IRL Press, Oxford (1995).

For example, methods for distinguishing wild-type DNA from mutants containing a single nucleotide change are described in PCT Application WO 87/07646. The methods disclosed in PCT Application WO 87/07646 are incorporated herein by reference.

Briefly, oligonucleotides containing either the wild-type or mutant sequence are hybridized under stringent conditions to dried agarose gels containing target RNA or DNA digested with appropriate restriction endonuclease. An example of a suitable stringent condition includes a temperature of two or more degrees below the calculated T.sub.m of a perfect duplex. The oligonucleotide probe hybridizes to the target DNA or RNA detectably better when the probe and the target are perfectly complementary.

A particularly convenient method for assaying a single point mutation by means of oligonucleotides is described in Segev, PCT Application WO 90/01069. The methods disclosed in PCT Application WO 90/01069 are hereby incorporated by reference.

Briefly, two oligonucleotide probes for each wild-type or mutated strand being assayed are prepared. Each oligonucleotide probe is complementary to a sequence that straddles the nucleotides at the site of the genetic variation. Thus, a gap is created between the two hybridized probes.

The gap is filled with a mixture of a polymerase, a ligase, and the nucleotide complementary to that at the position to form a ligated oligonucleotide product. Either of the oligonucleotides or the nucleotide tilling the gap may be labelled by methods known in the art.

The ligated oligonucleotide product can be amplified by denaturing it from the target, hybridizing it to additional oligonucleotide complement pairs, and filling the gap again, this time with the complement of the nucleotide that filled the gap in the first step.

The oligonucleotide product can be separated by size and the label is detected by methods known in the art.

Mutations may also be detected if they create or abolish restriction sites; see Baker et al. Science 244, 217-221 (1989). Some additional examples of the use of restriction analysis to assay point mutations are given in Weinberg et al, U.S. Pat. No. 4,786,718 and Sands, M. S. and Birkenmeier. E. H., Proc. Natl. Acad. Sci. USA 90:6567-6571 (1993).

For example, point mutations can be detected by means of single-strand conformation analysis of polymerase chain reaction products (PCR-SSCP). This method is described in Orita, M. et al., Proc. Natl. Acad. Sci. USA 86:2766-2770 (1989), Suzuki, Y. et al., Oncogene 5:1037-1043 (1990), and Sarkar, F. H. et al., Diagn. Mol. Pathol. 4:266-273 (1995).

Some additional methods for distinguishing wild-type DNA and its mutants are described by De Ley et al., J. Bacteriol. 101:738-754 (1970); Wood et al., Proc. Natl. Acad. USA 82:1585-1588 (1985); Myers et al., Nature 313:495-497 (1985); and Myers et al., Science 230:1242-1246 (1985).]

The presence of a mutation in the nucleic acid sequence of Nell1 compared to a wild-type Nell1 nucleic acid sequence indicates presence of a congenital heart defect in a mammal. In another aspect, the presence of a mutation in the nucleic acid sequence of Nell1 compared to a wild-type Nell1 nucleic acid sequence indicates an increased risk in a mammal for a congenital heart defect. No mutation in the nucleic acid sequence of Nell1 typically indicates that the mammal does not have a congenital heart defect or is not at an increased risk for a congenital heart defect. The nucleic acid sequence of Nell1 is highly conserved across species. Therefore, as used herein, the term “wild-type” Nell1 can be from any species. Thus, in one embodiment, the nucleic acid sequence of Nell1 (i.e., from the biological sample) is compared to the wild-type Nell1 nucleic acid molecule from the same species. In another embodiment, the nucleic acid sequence of Nell1 (i.e., from the biological sample) is compared to the wild-type Nell1 nucleic acid molecule from another species.

The term “mutation” as used herein is any alteration in the Nell1 nucleic acid sequence that alters function or expression of Nell1 gene products, such as mRNA and the encoded protein. Thus, degenerate sequences of the nucleic acid sequence of wild-type Nell1 (e.g., SEQ. ID. No: 2) are not considered to be mutations.

The mutation can occur anywhere in the nucleic acid sequence of Nell1. For example, the mutation can be in the coding and non-coding regions (e.g., promoter, introns, or untranslated regions, etc.) of Nell1. A mutation occurring in a regulatory region of the Nell1 gene, for example, can lead to loss or a decrease of expression of the mRNA, or can abolish proper mRNA processing leading to a decrease in mRNA stability or translation efficiency.

The mutation can be a deletion, substitution, insertion, rearrangement, point mutation, duplication, etc., and combinations thereof The deletion can, for example, be of the entire Nell1 gene, or only a portion of the gene. Alternatively, the mutation can result in, for example, a stop codon, frameshift, amino acid substitution, etc. For example, the mutation can be a single base change in the coding region of Nell1 (T→A) that results in the conversion of a cysteine codon to a premature stop codon (TGT→TGA). This specific mutation truncates the 810 amino acid Nell1 polypeptide at amino acid residue number 502.

Alternatively, since mutations in the Nell1 nucleic acid sequence can result in reduced levels of Nell1 nucleic acid molecules (e.g., mutations in the regulatory regions of the gene or mutations in the coding or non-coding regions that affect RNA stability), mutations in Nell1 nucleic acid molecules can be assessed by evaluating whether Nell1 nucleic acid molecules are present at reduced levels in a biological sample.

Determining whether Nell1 nucleic acid molecules are present at reduced levels in a biological sample may be accomplished by any method known in the art. Some examples include, extracting and/or amplifying mRNA from the biological sample and quantifying it by such methods as electrophoresis and staining, or alternatively by means of Southern blot and the use of suitable probes, Northern blot and use of probes specific for the Nell1 mRNA or its corresponding cDNA, real-time quantitative PCR etc.

Similarly, the level of the corresponding cDNA to Nell1 mRNA can also be quantified by means of the use of conventional techniques. For example, cDNA is synthesized by means of reverse transcription (RT) of the corresponding Nell1 mRNA followed by amplification and quantification of the cDNA amplification product.

In one embodiment, determination of the level of Nell1 nucleic acid molecules in a biological sample is quantitative. The quantitative assays for determining this amount may, for example, use known quantities (i.e., standards) of Nell1 nucleic acid molecules. These standards may be used to generate a standard curve that relates a concentration of Nell1 nucleic acid molecules to the quantity of a detectable signal. The detectable signal can be, for example, the quantity of light emitted or absorbed (e.g., optical density, such as fluorescence intensity) or quantity of radioactivity emitted (e.g., radioactive counts per minute).

For example, a graph of known concentrations of Nell1 nucleic acid molecules versus optical density, fluorescence intensity or radioactive counts may be used to calculate the amount (e.g., concentration) of Nell1 nucleic acid molecules in a biological sample. The amount of Nell1 nucleic acid molecules detected in a sample using a quantitative assay is typically compared to the amount of Nell1 nucleic acid molecules in a control sample (i.e., background amount). A control sample is typically a sample from a mammal with no medical history of a congenital heart defect and has no CHD detectable by current diagnostic techniques. For instance, a chip-based method, such as a microarray, can be utilized to determine the level of Nell1 nucleic acid molecules in a biological sample.

It is not, however, necessary to generate a standard curve or to calculate the amount of Nell1 nucleic acid molecules in a biological sample. Alternatively; the quantity of the detectable signal (e.g., light absorbed or emitted, or radioactivity emitted) from a biological sample to that of a control sample (i.e., background signal) may be used as a measure of the amount of Nell1 nucleic acid molecules in a biological sample relative to the control sample. The quantity of detectable signal is indicative of the amount of Nell1 nucleic acid molecules present in a biological sample since an increase in optical density or radioactive counts correlate with an increase in the concentration of Nell1 nucleic acid molecules. Accordingly, the quantity of detectable signal may be used as a measure of the amount of Nell1 nucleic acid molecules in a biological sample.

It is not necessary to determine the background amount or the quantity of background signal each time an assay is conducted. It is well known in the art to compare the amount of Nell1 nucleic acid molecules or the quantity of detectable signal obtained as a measure of the amount of Nell1 nucleic acid molecules in the test sample to that of a previously determined background amount or background signal.

In one aspect, an amount of Nell1 nucleic acid molecules significantly lower than that of a control indicates the presence of a congenital heart defect in the mammal. In another aspect, an amount of Nell1 nucleic acid molecules significantly lower than that of a control indicates an increased risk for a congenital heart defect in a mammal. (It is understood that, as used herein, the amount of Nell1 nucleic acid molecules may be indicated by the quantity of the detectable signal.) The risk for developing a congenital heart defect is increased by at least about 10% compared to a mammal that does not contain a mutation in the nucleic acid sequence of Nell1, more typically, the risk in increased by about 25%, more typically increased by about 50%, and even more typically increased by about 75%.

If the amount of Nell1 nucleic acid molecules in the control is a mean value, and the standard deviation of the mean value is known, or can be calculated, an amount is considered to be significantly lower if the amount is at least two standard deviations lower than the mean value of the control. If the standard deviation is not known, and cannot be calctilated, an amount is significantly lower if the amount is at least about 10%. preferably at least about 25%, more preferably at least about 50%, even more preferably at least about 75%, and most preferably at least about 100% lower than that of the control.

### Method for Detecting, a Congenital Heart Defect, or Increased Risk of a Congenital Heart Defect, by Assessing Nell1 Protein

In another aspect, the invention provides a method for detecting a congenital heart defect in a mammal by assessing Nell1 protein. In yet another aspect, the invention provides a method for detecting increased risk for a congenital heart defect. The first step in these methods is to provide a biological sample. The biological sample can he obtained, in the same laboratory in which the method is performed, or in another center and later sent to the laboratory for study. The biological sample contains a Nell1 protein. Examples of biological sample that contain Nell1 protein include those samples discussed above (e.g., blood cells, saliva, epithelial cells, fetal cells, placental cells, amniotic fluid, and chorion villus sample).

The Nell1 protein is assessed for the presence of a mutation. Assessing the presence of a mutation in a Nell1 protein can be determined by any method known to those skilled in the art. An example of a suitable method is, for example, sequencing the Nell1 protein from the biological sample and comparing the sequence to the amino acid sequence of wild type Nell1 protein (e.g., SEQ. ID. No: 1). The detection of a mutation indicates a congenital heart defect, or an increased risk for a congenital heart defect, in the mammal. No mutation in the protein sequence of Nell1 typically indicates that the mammal does not have a congenital heart defect or is not at an increased risk for a congenital heart defect. As stated above, the term “mutation” as used herein is any alteration in the Nell1 amino acid sequence that alters function or expression of the protein. The presence of a mutation in a Nell1 protein in the biological sample indicates a congenital heart defect in the mammal.

The amino acid sequence of Nell1 is highly conserved across species. For example, the mouse Nell1 protein shares about 93% sequence identity with the human Nell1 protein, which, in turn, shares about 90% sequence identity with the rat Nell1 protein. Therefore, as used herein, the term “wild-type” Nell1 can be from any species. Thus, in one embodiment, the amino acid sequence of Nell1 (i.e., from the biological sample) is compared to the wild-type Nell1 amino acid sequence from the same species. In another embodiment, the amino acid sequence of Nell1 (i.e., from the biological sample) is compared to the wild-type Nell1 amino acid sequence from another species.

Alternatively, since mutations can result in the reduction of Nell1 protein levels, presence or susceptibility for CHDs can be screened by evaluating whether Nell1 protein is present at reduced levels in a biological sample.

Determining whether Nell1 protein is present at reduced levels in a biological sample may be accomplished by any method known in the art. Some examples include immunoassays such as, for example, an ELISA (Current Protocols in Immunology, Wiley Intersciences, New York, 1999) and a standard blot assay (Towbin et al., 1979 and Towbin et al., 1984). These assays are normally based on incubating a sample containing Nell1 protein with an antibody specific for Nell1, and detecting the presence of a complex between the antibody and the protein. For example, the antibody is preferably immobilized prior to detection and is referred to as a capture antibody. For the purposes of this invention, the capture antigen is typically Nell1. Immobilization may be accomplished by directly binding the capture antibody to a solid surface, such as a microtiter well. If Nell1 protein is present in the sample, the protein will bind to the capture antibody.

A second antibody is added that binds specifically to an epitope of Nell1 protein in the sample. The second antibody may be labeled by methods known in the art. The secondary antibody may, for example, be radiolabeled or enzymatically labeled. Preferably, the labeled second antibody is enzymaticaly labeled to provide, for example, visual or photometric analysis. Examples of such enzymatic labels include, for example, horse radish peroxidase and alkaline phosphatase. Some examples of photometric instruments that may be used for analysis include, for example, a spectrophotometer and an ELISA plate reader.

In general, it is desirable to provide incubation conditions sufficient to cause binding of as much Nell1 protein present in the sample as possible. The specific concentrations of labeled second antibodies, the temperature and time of incubation, as well as other such assay conditions, can be varied, depending upon various factors including the concentration of Nell1 protein in the sample, the nature of the sample and the like. Those skilled in the art will be able to determine operative and optimal assay conditions for each determination by employing routine experimentation.

In one embodiment, determination of the level of Nell1 protein in a biological sample is quantitative. The quantitative assays for determining this amount may, for example, use known quantities (i.e., standards) of Nell1 protein. These standards may be used to generate a standard curve that relates a concentration of Nell1 protein to the quantity of a detectable signal. The detectable signal can be, for example, the quantity of light emitted or absorbed (e.g., optical density, such as fluorescence intensity) or quantity of radioactivity emitted (e.g., radioactive counts per minute).

For example, a graph of known concentrations of Nell1 protein versus optical density or radioactive counts may be used to calculate the amount (e.g., concentration) of Nell1 protein in a biological sample. The amount of Nell1 protein detected in a sample using a quantitative assay is typically compared to the amount of Nell1 protein in a control sample (i.e., background amount). A control sample is typically a sample from an mammal with no medical history a congenital heart defect and standard clinical tests did not reveal structural or functional heart defects. For instance, a chip-based method, such as a microarray, can be utilized to determine the level of Nell1 protein in a biological sample

It is not, however, necessary to generate a standard curve or to calculate the amount of Nell1 protein in a biological sample. Alternatively, the quantity of the detectable signal (e.g., light absorbed or emitted, or radioactivity emitted) from a biological sample to that of a control sample (i.e., background signal) may be used as a measure of the amount of Nell1 protein in a biological sample relative to the control sample. The quantity of detectable signal is indicative of the amount of Nell1 protein present in a biological sample since an increase in optical density or radioactive counts correlate with an increase in the concentration of Nell1 protein. Accordingly, the quantity of detectable signal may be used as a measure of the amount of Nell1 protein in a biological sample.

It is not necessary to determine the background amount or the quantity of background signal each time an assay is conducted. It is well known in the art to compare the amount of Nell1 protein or the quantity of detectable signal obtained as a measure of the amount of Nell1 protein in the test sample to that of a previously determined background amount or background signal.

In one aspect, an amount of Nell1 protein significantly lower than that of a control indicates the presence of a congenital heart defect in the mammal. In another aspect, an amount of Nell1 protein significantly lower than that of a control indicates an increased risk for a congenital heart defect in a mammal. (It is understood that, as used herein, the amount of Nell1 protein may be indicated by the quantity of the detectable signal.) The risk for developing a congenital heart defect is increased by at least about 10%. more typically, the risk in increased by about 25%, more typically increased by about 50%, and even more typically increased by about 75%.

If the amount of Nell1 protein in the control is a mean value, and the standard deviation of the mean value is known, or can be calculated, an amount is considered to be significantly lower if the amount is at least two standard deviations lower than the mean value of the control. If the standard deviation is not known, and cannot be calculated, an amount is significantly lower if the amount is at least about 10%, preferably at least about 25%. more preferably at least about 50%, even more preferably at least about 75%, and most preferably at least about 100% lower than that of the control.

Mutations can result in alteration of Nell1 protein activity, even if the protein levels are unchanged. In some instances mutations detected by DNA and/or protein sequencing need further assessment as to its impact in Nell1 function (e.g. conservative amino acid substitutions). Confirmation of the adverse effect in Nell1 protein function can be evaluated by cell based assays or methods that will measure protein-protein binding. One example is the use of techniques to examine binding ability of the extracted Nell1 to protein kinase C. A second example is the addition of the extracted Nell1 protein to a cell culture of precursor cells that Nell1 normally stimulates to differentiate (e.g. osteoblast or cardiomyocyte precursor cells). Inability of the protein to trigger differentiation will indicate impairment of Nell1 protein activity.

### Methods of Treating a Congenital Heart Defect

In another aspect, the invention provides a method for treating a congenital heart defect in a mammal in need thereof. The method comprises administering an effective amount of a Nell1 protein to the mammal. Any congenital heart defect can be treated in accordance with the method of the invention. Examples of congenital heart defects include those described above.

The Nell1 protein useful in the methods for treating a congenital heart defect can comprise a polypeptide having the same amino acid sequence as Nell1 protein derived from nature (e.g., wild-type Nell1 protein), a recombinant Nell1 protein, a homolog thereof, or fragments thereof. Accordingly, a “Nell1 protein” as used herein, also refers to recombinants, homologs and fragments thereof.

As mentioned above, since the amino acid sequence of Nell1 protein is highly conserved across species, the naturally occurring amino acid sequence of Nell1 protein can be from any animal. For example, the Nell1 protein can be human Nell1, rat Nell1, or mouse Nell1.

The structure of Nell1 proteins has been characterized (see, e.g., Kuroda et al., 1999a: Kuroda et al., 19991). Desai et al., 2006). For example, the mouse Nell1 protein (SEQ ID NO: 5) is a protein of 810 amino acids, having a secretion signal peptide (amino acids 1 to 16), an N-terminal TSP-like module (amino acids #29 to 213), a Laminin G region (amino acids #86 to 210), von Willebrand factor C domains (amino acids #273 to 331 and 699 to 749), and a Ca²⁺-binding EGF-like domains (amino acids #549 to 586).

Homologs of Nell1 protein include, for example, a substitution mutant, a mutant having an addition or insertion, or a deletion mutant of the protein. Substitutions in a sequence of amino acids are preferably with equivalent amino acids. Groups of amino acids known to be of equivalent character are listed below:

| | |
|---|---|
| (a) | Ala(A), Ser(S), Thr(T), Pro(P), Gly(G); |
| (b) | Asn(N), Asp(D), Glu(E), Gln(Q); |
| (c) | His(H), Arg(R). Lys(K); |
| (d) | Met(M), Leu(L), Ile(I), Val(V); |
| and | |
| (e) | Phe(F), Tyr(Y), Trp(W). |

Any substitutions, additions, and/or deletions in an amino acid sequence are permitted provided that the Nell1 protein is functional. An amino acid sequence that is substantially identical to another sequence, but that differs from the other sequence by means of one or more substitutions, additions, and/or deletions, is considered to be an equivalent sequence.

In order to compare a first amino acid to a second amino acid sequence for the purpose of determining homology, the sequences are aligned so as to maximize the number of identical amino acid residues. The sequences of highly homologous proteins can usually be aligned by visual inspection. If visual inspection is insufficient, the amino acid molecules may be aligned in accordance with methods known in the art. Examples of suitable methods include those described by George, D. G. et al., in Macromolecular Sequencing and Synthesis, Selected Methods and Applications, pages 127-149, Alan R. Liss, Inc. (1988), such as formula 4 at page 137 using a match score of 1, a mismatch score of 0, and a gap penalty of −1. Alternatively, any computational method known to those skilled in the art used for aligning protein sequences to access identity can be utilized.

Preferably, less than 15%, more preferably less than 10%, and still more preferably less than 5% of the number of amino acid residues in the sequence of Nell1 are different (i.e., substituted for, inserted into, or deleted from). More preferably still, less than 3%, yet more preferably less than 2% and optimally less than 1% of the number of amino acid residues in a sequence are different from those in a naturally occurring sequence.

Preferably, the substitutions, additions, and/or deletions are not made in the conserved regions of the protein or in the functional domain of the protein. Examples of conserved regions of Nell1 protein include the secretory signal, Willebrand like domain, thrombospondin-like domains and laminin-like domains. Examples of functional domains of Nell1 protein include the EGF-like domains. Thus, substitutions, additions, and/or deletions in the non-conserved and/or non-functional regions of the protein can typically be made without affecting the function of Nell1 protein.

A Nell1 protein further includes Nell1 protein fragments that retain the ability to promote repair of a congenital heart defect. In one embodiment, the Nell1 protein fragment contains one or more of the conserved regions and/or functional domains of the protein. For example, the Nell1 protein, fragments can comprise the EGF like domains and/or the von Willebrand like domain of Nell1 protein. The term “fragment” as used herein typically has a maximum length of about 800 amino acid residues, more typically a maximum length of about 700 amino acid residues, even more typically a maximum length of about 600 amino acid residues, and yet more typically a maximum length of about 500 amino acid residues. A fragment of Nell1 protein generally has a minimum length of about 10 amino acid residues, more generally a minimum length of about 20 amino acid residues, even more generally a minimum length of about 30 amino acid residues, and yet more generally a minimum length of about 40 amino acid residues.

Once a Nell1 protein homolog or Nell1 protein fragment is made, such protein can be tested to determine whether it retains substantially the activity or function of a wild type Nell1 protein. For example, the ability of a Nell1 homolog or fragment to bind PKC beta can be tested. Suitable assays for assessing the binding of Nell1 to PKC beta is described in e.g., Kuroda et al. (Biochemical Biophysical Research Comm. 265: 752-757 (1999b)). In addition, the ability of a Nell1 protein homolog or fragment to stimulate differentiation of precursor cells that are stimulated or activated by Nell1 into more mature or differentiated states can be tested (e.g. precursor cells to osteoblast. chordrocyte, cardiomyocyte, skeletal satellite, neuronal, endothelial cells etc.). Nell1-induced cell differentiation can be assessed cellularly (histology) and molecularly (expression of skeletal muscle-specific proteins or extracellular matrix materials). Still further, a Nell1 protein homolog or fragment can be tested for its ability to drive osteoblast precursors to mature bone cells, by detecting expression of late molecular bone markers or mineralization (i.e., calcium deposits). By comparing the activity of a Nell1 protein homolog or fragment with that of a wild type Nell1 protein in one or more of the assays such as those described above, one can determine whether such homologs or fragments retain substantially the activity or function of a wild-type Nell1 protein.

In yet another aspect, the invention provides a method for treating a congenital heart defect in a mammal in need thereof. The method comprises administering to the mammal a nucleic acid coding for a Nell1 protein. Any nucleic acid sequence that encodes for Nell1 protein can be used in the methods of the present invention. Suitable nucleic acid molecules encoding Nell1 protein for use in the methods of the present invention include nucleic acid molecules having a nucleotide sequence as set forth in SEQ. ID. NOs: 2, 4 and 6. The nucleic acid molecules can be incorporated into recombinant vectors suitable for use in gene therapy.

Examples of vectors suitable for use in gene therapy may be any vector that comprises a nucleic acid sequence capable of expressing the Nell1 protein in a mammal, especially a human, in need of such therapy. The suitable vector may be for example a viral vector, such as an adenovirus vector or an adeno-associated virus (AAV) vector. See for example: Ledley 1996. Pharmaceutical Research 13:1595-1614 and Verma et al. Nature 1997, 387:239-242. Other examples of suitable vectors include plasmids, such as PAC. YAC (yeast artificial chromosome) and BAC (bacterial artifical chromosome).

Alternatively, treatment of congenital heart defects with a Nell1 protein can also be performed via naturally occuring or modified (differentiated in vitro or genetically modified) cells that express high levels of Nell1. For example, Nell1 expressing cells are found in the epicardial, endocardial and pericardial layers of the heart. Cells in the inner linings of blood vessels also abundantly express Nell1 protein. These specific cell populations can, for example be isolated, established and expanded in vitro and concentrated. The concentrated cell population can be introduced to a patient's heart via methods known to those skilled in the art for delivering therapeutic cells (e.g. Stem cells delivered by catheter based methods, cell-infused biopatches, intramyocardial injections etc.). Modification of pericardial cells by gene therapy to express high levels of Nell1 protein to the underlying heart muscle is another potential cell-based method.

The Nell1 protein or nucleic acid molecule is administered to a mammal in need thereof. The mammal may be a farm animal, such as a goat, horse, pig, or cow; a pet animal, such as a dog or cat; a laboratory animal, such as a mouse, rat, or guinea pig; or a primate, such as a monkey, orangutan, ape, chimpanzee, or human. In a preferred embodiment, the mammal is a human.

Mammals in need of the treatment methods in accordance with the invention include those mammals that have, or have been diagnosed, with a congenital heart defect. Another example of mammals in need include those mammals that have a mutation in the Nell1 nucleic acid sequence or Nell1 amino acid sequence.

### Administration

The Nell1 protein or nucleic acid molecule can be incorporated in a pharmaceutical composition suitable for use as a medicament, for human or animal use. The pharmaceutical compositions may be for instance, in an injectable formulation, a liquid, cream or lotion for topical application, an aerosol, a powder, granules, tablets, suppositories or capsules, such as for instance, enteric coated capsules etc. The pharmaceutical compositions may also be delivered in or on a lipid formulation, such as for instance an emulsion or a liposome preparation. The pharmaceutical compositions are preferably sterile, non-pyrogenic and isotonic preparations, optionally with one or more of the pharmaceutically acceptable additives listed below.

Pharmaceutical compositions of Nell1 protein or nucleic acid molecule are preferably stable compositions which may comprise one or more of the following: a stabilizer, a surfactant, preferably a nonionic surfactant, and optionally a salt and/or a buffering agent. The pharmaceutical composition may be in the form of an aqueous solution, or in a lyophilized form.

The stabilizer may, for example, be an amino acid, such as for instance. glycine: or an oligosaccharide, such as for example, sucrose, tetralone, lactose or a dextram. Alternatively, the stabilizer may be a sugar alcohol, such as for instance, mannitol; or a combination thereof. Preferably the stabilizer or combination of stabilizers constitutes from about 0.1% to about 10% weight for weight of the Nell1 protein.

The surfactant is preferably a nonionic surfactant, such as a polysorbate. Some examples of suitable surfactants include Tween20, Tween80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal. Preferably the salt or buffering agent is present at a roughly isotonic concentration of about 150 mM to about 300 mM.

The pharmaceutical composition comprising Nell1 protein or nucleic acid molecule may additionally contain one or more conventional additive. Some examples of such additives include a solubilizer such as for example, glycerol; an antioxidant such as for example, benzalkonium chloride (a mixture of quaternary ammonium compounds, known as “quats”), benzyl alcohol, chloretone or chlorobutanol; anaesthetic agent such as for example a morphine derivative; or an isotonic agent etc., such as described above. As a further precaution against oxidation or other spoilage, the pharmaceutical compositions may be stored under nitrogen gas in vials sealed with impermeable stoppers.

An effective amount of the Nell1 protein or nucleic acid molecule, preferably in a pharmaceutical composition, may be administered to a human or an animal in need thereof by any of a number of well-known methods. For example, the Nell1 protein or nucleic acid molecule may be administered systemically or locally, for example by injection.

The systemic administration of the Nell1 protein or nucleic acid molecule may be by intravenous, subcutaneous, intraperitoneal, intramuscular, intrathecal or oral administration.

In another embodiment, the Nell1 protein can be administered by a cell-based gene therapy. For example, allogeneic or xenogenic donor cells are genetically modified in vitro to express and secrete Nell1 protein. The genetically modified donor cells are then subsequently implanted into the mammal in need for delivery of Nell1 protein in vivo. Examples of suitable cells include, but are not limited to, endothelial cells, epithelial cells, fibroblasts, cardiomyoblasts, stem cells, such as adult stern cells, embryonic stem cells, and cord blood stem cells.

Alternatively, the genetically modified donor cells can be incorporated into a matrix containing an appropriate microenvironment to maintain, for a given time, the viability and growth of the genetically modified donor cells. The matrix can be applied to, for example, the myocardium. Expression and secretion of Nell1 by the genetically modified donor cells promotes healing of the myocardium. After the wound is healed, the matrix can be removed. Examples of suitable matrices include, but are not limited to, collagen matrix, patches, and hydrogels.

An effective amount of a pharmaceutical composition of the invention is any amount that is effective to achieve its purpose. The effective amount, usually expressed in mg/kg can be determined by routine methods during pre-clinical and clinical trials by those of skill in the art.

The Nell1 protein may be prepared by methods that are well known in the art. One such method includes isolating or synthesizing DNA encoding the Nell1 protein, and producing the recombinant protein by expressing the DNA, optionally in a recombinant vector, in a suitable host cell or cell-free transcription and translation systems. Suitable methods for synthesizing DNA are described by Caruthers et al. 1985. Science 230:281-285 and DNA Structure, Part A: Synthesis and Physical Analysis of DNA, Lilley, D. M. J. and Dahlberg. J. E. (Eds.), Methods Enzymol., 211, Academic Press, Inc., New York (1992). Examples of suitable Nell1 nucleic acid sequences include SEQ. ID. NOs: 2, 4, and 6.

The Nell1 protein may also be made synthetically, i.e. from individual amino acids, or semisynthetically, i.e. from oligopeptide units or a combination of oligopeptide units and individual amino acids. Suitable methods for synthesizing proteins are described by Stuart and Young in “Solid Phase Peptide Synthesis,” Second Edition, Pierce Chemical Company (1984), Solid Phase Peptide Synthesis, Methods Enzymol., 289, Academic Press. Inc, New York (1997). Examples of suitable Nell1 amino acid sequences include SEQ. ID. NOs: 1, 3, 5, homologs thereof, and fragments thereof.

This invention is further illustrated by the following examples, which are not to be construed in any way as imposing limitations upon the scope thereof. The terms and expressions which have been employed in the present disclosure are used as terms of description and not of limitation, and there is no intention in the use of such terms and expressions of excluding any equivalents of the features shown and described or portions thereof. It is to be understood that various modifications are considered to be included within the scope of the invention. All the publications mentioned in the present disclosure are incorporated herein by reference.

### Examples

### Example 1

### Nell1 is Expressed in the Developing Heart

Expression of the Nell1 gene in the development of the mammalian heart was detected in mouse fetuses at 18.5 days of gestation. Fetal mouse hearts were dissected from fetuses and quickly preserved in RNAlater solution to preserve the tissues. RNA was extracted by homogenization of pooled mouse hearts in guanidine isothiocyanate solution and subsequent RNA extractions with the phase lock gel tube system [Eppendorf; Phenol/chloroform isoamyl alchohol extractions of the aqueous layer and ethanol precipitation). cDNA was synthesized from the RNA samples by reverse transcription PCR using a commercial cDNA synthesis kit (Ambion). The presence of Nell1 cDNA was detected by PCR amplification of three overlapping segments of the coding region (827, 866 and 798 bp) using primers designed based on the published gene sequence (FIG. 1). All three expected Nell1 segments were amplified from the fetal RNA samples and were confirmed by direct DNA sequencing of the amplified products. In addition, alternative segments at the 5′ and 3′ ends (starting and ending segments) were detected while the middle segment was unique. This suggested that alternative Nell1 protein products are present in the heart and that the variation from the full-length cDNA are at the front and end of the coding region.

### Example 2

### Congenital Heart Defects are Associated with the Loss of Function of the Nell1 Protein

Congenital heart defects associated with the loss of function of the Nell1 protein were determined by examining the hearts of Nell1 mutant mouse fetuses at E15.5 (mid-gestation) and 18.5 days of gestation and comparing them to control littermates. The mutant fetuses are homozygous (2 mutant copies) for the Nell1^{6R} mutation. Fetuses were collected and fixed in buffered formalin overnight and transferred to 70% ethanol solution. The thoracic region was removed, embedded in paraffin, sectioned and mounted in slides and then stained with haematoxylin-eosin. Hearts were examined using light microscopy and differences between mutant and normal fetuses were noted. Observations of valve defects (FIGS. 2 and 3) from 21 E15.5 fetuses (9 mutant, 12 normal) and 15 E18.5 fetuses (7 mutant, 8 normal). The following defects were observed: enlarged valves, decreased ventricular chamber sizes, underdeveloped atrial chambers, immature architecture of the chordae tendinae, abnormal number and shape of valve leaflets.

### Example 3

### Prenatal Screening

Prenatal screening during mid-gestation are conducted by accepted methods such as amniocentesis, chorion villus sampling or any techniques that permit collection of fetal cells. Fetuses with a family history of heart defects, especially when associated with bone and skeletal defects, are high-priority candidates for Nell1 screening.

Loss-of-function mutations are assayed in DNA or RNA (cDNA generated from RNA) extracted from fetal cells. Presence of Nell1 mutation(s) that affect protein structure and function identifies fetuses with high-risk susceptibility for CHD.

Upon identification of such Nell1 mutations, the following follow-up clinical decisions can be made. More frequent electronic fetal heart monitoring during the entire gestation process can be conducted. In addition, high resolution imaging of fetal heart structure and function can be performed. For example, in utero 3D ultrasonographic imaging at mid to late gestation can reveal both structural and functional anomalies of the specific heart structures that are influenced by Nell1 activity during development such as the myocardium, vessels, valves and chambers. Thus, an early decision for caesarean and/or premature delivery can be made. Infants with CHDs will be at great risk for death or complications in a natural delivery process, hence Nell1 mutation screening can potentially identify at an early stage, fetuses that are at high risk. Fetuses that have Nell1 mutations and CHD(s) are identified early enough for treatment during the neonatal or early infancy period.

### Example 4

### Treatment of Congenital Heart Disease 1

Young patients (infants and children) diagnosed with congenital heart defects (e.g., valve defects) are treated with the Nell1 protein or gene by using any of several cardiac-specific delivery systems. In addition, there are established surgical procedures to replace or repair heart valves, Nell1 protein can be delivered to the area around the developing heart valve and applied as part of a device, absorbable gel matrix or other biomatrix. The Nell1-containing device or biomaterial can be introduced into the valve area using catheterization techniques that will permit delivery into the heart valve area and released for proper placement.

Children that are found to have Nell1 loss-of-function mutations and/or CHDs but are asymptomatic for heart function defect when examined with current detection methods (valve problems and anomalies of the atrial chambers can be detected by cardiac catheterization, Xray, Doppler ultrasound, electrocardiography (ECG). MRI and a transesophageal ECG), can be placed in a high risk category for manifestation of future cardiac anomalies. It is important to identify and monitor these patients because certain CHDs can predispose towards future valve calcification and stenosis (Sabel et al, 1999). In addition, underlying CHDs that were not detected early in life can be revealed in the future under certain conditions that impose cardiac stress (e.g. pregnancy, sports etc.) on the individual and when serious enough can result in sudden cardiac deaths (Fabre and Sheppard 2006). These asymptomatic patients are recommended for more frequent cardiac function monitoring, and upon onset of the defects, are then treated with the appropriate surgical method or drug treatment.

### Example 5

### Treatment of Cardiomyopathy

In CHDs where the defect is cardiomyopathy of the heart muscle, Nell1 protein can be delivered to the heart during neonatal or juvenile stage to aid in the strengthening or developing of the heart muscle. In this application, Nell1 can be delivered to the heart muscle by the following (but not exclusively) variety of methods: drug delivery from coronary stents, infusion into the pericardial space, ultrasonic methods, biogels or matrices, nanoparticles (Mayer and Bekeredjian 2008; Bekeredjian et al 2005. Xiao et al, 2008; Scott et al 2008; Esaki et at 2007). Improvement of cardiac function by Nell1 treatment is ascertained by echocardiography, EKG and other routine clinical methods for examining heart function.

1. A method for detecting a congenital heart defect in a mammal, the method comprising:
(i) providing a biological sample from the mammal, wherein said biological sample comprises a Nell1 nucleic acid molecule, and
(ii) assessing said Nell1 nucleic acid molecule for the presence of a mutation;
whereby the presence of a mutation in the Nell1 nucleic acid molecule indicates presence of a congenital heart defect in the mammal.

2. A method according to claim 1, wherein the sample is a prenatal sample.

3. A method according to claim 1, wherein the sample is a neonatal sample.

4. A method according to claim 1, wherein the sample is an amniotic sample.

5. A method according to claim 1, wherein the sample is a chorion villus sample.

6. A method according to claim 1, wherein mammal is a human.

7. A method according to claim 1, wherein the sample is a blood sample.

8. A method according to claim 1, wherein the sample is a buccal sample.

9. A method according to claim 1, wherein presence of a mutation in the Nell1 nucleic acid molecule is determined by assessing the level of Nell1 nucleic acid molecules in the biological sample.

10. A method for detecting increased risk for a congenital heart defect in a mammal, the method comprising:
(i) providing a biological sample from the mammal, wherein said biological sample comprises a Nell1 nucleic acid molecule, and
(ii) assessing said Nell1 nucleic acid molecule for the presence of a mutation;
whereby the presence of a mutation in the Nell1 nucleic acid molecule indicates increased risk for a congenital heart defect in the mammal.

11. A method according to claim 10, wherein presence of a mutation in the Nell1 nucleic acid molecule is determined by assessing the level of Nell1 nucleic acid molecules in the biological sample.

12. A method for detecting a congenital heart defect in a mammal, the method comprising:
(i) providing a biological sample for the mammal, wherein said biological sample comprises Nell1 protein, and
(ii) assessing said Nell1 protein for the presence of a mutation;
whereby the presence of a mutation in the Nell1 protein indicates presence of a congenital heart defect in the mammal.

13. A method according to claim 12, wherein presence of a mutation in the Nell1 protein is determined by assessing the level of Nell1 protein in the biological sample.

14. A method for detecting increased risk for a congenital heart defect in a mammal, the method comprising:
(i) providing a biological sample for the mammal, wherein said biological sample comprises Nell1 protein, and
(ii) assessing said Nell1 protein for the presence of a mutation;
whereby the presence of a mutation in the Nell1 protein indicates increased risk for a congenital heart defect in the mammal.

15. A method according to claim 14, wherein presence of a mutation in the Nell1 protein is determined by assessing the level of Nell1 protein in the biological sample.

16. A method for treating a congenital heart defect in a mammal in need thereof, the method comprising administering an effective amount of Nell1 protein to the mammal.

17.-19. (canceled)

20. A method according to claim 16, wherein the Nell1 protein is delivered by a cell.

21. A method according to claim 16, wherein the Nell1 protein is human Nell1 protein.

22. A method according to claim 16, wherein the mammal is a human.

23. A method according to claim 16, wherein the Nell1 protein is administered systemically.

24. A method according to claim 16, wherein the Nell1 protein is administered locally.

25. A method according to claim 24, wherein local administration is by injection.

26. A method for treating a congenital heart defect in a mammal in need thereof, the method comprising administering to the mammal a nucleic acid coding for a Nell1 protein.


SEQUENCE LISTING (6 sequences)

SEQ ID NO: 1 - DNA, 20 bp, Artificial Sequence
  primer
ggttctgtgtgtgcaccgcc

SEQ ID NO: 2 - DNA, 21 bp, Artificial Sequence
  primer
ggagtcttggtccctgtagag

SEQ ID NO: 3 - DNA, 20 bp, Artificial Sequence
  primer
cctgccaagtgagtgggctg

SEQ ID NO: 4 - DNA, 21 bp, Artificial Sequence
  primer
gcactcaacgaatccctctgc

SEQ ID NO: 5 - DNA, 20 bp, Artificial Sequence
  primer
tcacaggaagccactgtgag

SEQ ID NO: 6 - DNA, 21 bp, Artificial Sequence
  primer
gacacaccagatccacagagc